(19) United States
(12) Patent Application Publication
(10) Pub. No.: US 2023/0355421 A1
(43) Pub. Date: Nov. 9, 2023

(54) ARM IMMOBILIZING SLEEP GARMENT DEVICE

(71) Applicant: Nelson Howe, Denver, CO (US)

(72) Inventor: Nelson Howe, Denver, CO (US)

(21) Appl. No.: 18/222,914

(22) Filed: Jul. 17, 2023

Related U.S. Application Data
(63) Continuation-in-part of application No. 17/381,333, filed on Jul. 21, 2021, now Pat. No. 11,717,039.

(51) Int. Cl. A61F 5/37; A41D 1/04; A41D 13/12

(52) U.S. Cl. CPC A61F 5/3723; A41D 1/04; A41D 13/1245

(57) ABSTRACT

An arm immobilizing sleep garment device a vest having a chest, a neck, and a back. A mating panel is coupled to the chest of the vest. A glove has a palm and a plurality of finger sleeves such that the glove is configured for being worn on a hand. A mating piece is coupled to the glove and is complementary to the mating panel such that abutting the mating piece to the mating panel engages the glove to the vest. Thus, the glove and vest immobilize an arm extending from the hand in the glove.

### CROSS-REFERENCE TO RELATED APPLICATIONS

This is a continuation in part claiming the benefit under 35 U.S.C., Section 120 of U.S. application Ser. No. 17/381,333 filed Jul. 21, 2021.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to sleep garment devices and more particularly pertains to a new sleep garment device for immobilizing a user's arm while the user is sleeping. The device includes a vest which has a mating panel positioned on one side of a chest of the vest. A glove is provided that can be worn on the user's hand. A mating piece complementary to the mating panel is attached to the glove. The mating piece on the glove is matable to the mating panel on the chest of the vest. In this way the user's arm can be restrained while the user is sleeping during recovery from shoulder surgery.

### (2) Description of Related Art Including Information Disclosed Under 37 CFR 1.97 and 1.98

The prior art relates to sleep garment devices including a band that is wearable around a user's shoulders and a pair of gloves that are matable to the band for restraining the user's arms during post-surgical recovery. The prior art discloses a waist belt that is wearable around a user's waist and a wrist cuff that is matable to the waist belt for retraining the user's arm during post-surgical recovery. The prior art discloses a post surgical garment that includes a belt strap, an over-the-shoulder strap that is coupled to the belt strap and a wrist cuff that is attachable to the over-the-shoulder strap for immobilizing a user's arm. The prior art discloses a post surgical garment that includes a vest, a pair of loops each integrated into the vest and a pair of wrist cuffs that are extendable through respective loops for immobilizing a user's arms. The prior art discloses an immobilizing garment that includes a shirt and a plurality of loops each integrated into the shirt which can receive fingers on the user's hands for immobilizing the user's hands.

### BRIEF SUMMARY OF THE INVENTION

An embodiment of the disclosure meets the needs presented above by generally comprising a vest having a chest, a neck, and a back. A mating panel is coupled to the chest of the vest. A glove has a palm and a plurality of finger sleeves such that the glove is configured for being worn on a hand. A mating piece is coupled to the glove and is complementary to the mating panel such that abutting the mating piece to the mating panel engages the glove to the vest. Thus, the glove and vest immobilize an arm extending from the hand in the glove.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of an arm immobilizing sleep garment device according to an embodiment of the disclosure.

FIG. 2 is a back view of an embodiment of the disclosure.

FIG. 3 is a left side view of a vest of an embodiment of the disclosure.

FIG. 4 is a perspective in-use view of an embodiment of the disclosure.

FIG. 5 is a front view of an embodiment of the disclosure.

### DETAILED DESCRIPTION OF THE INVENTION

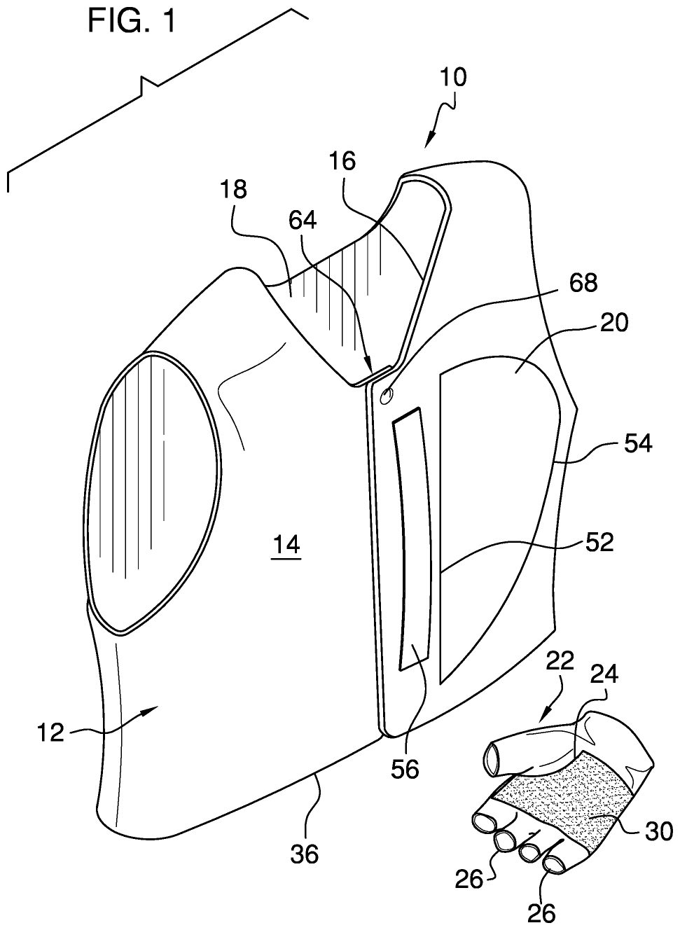

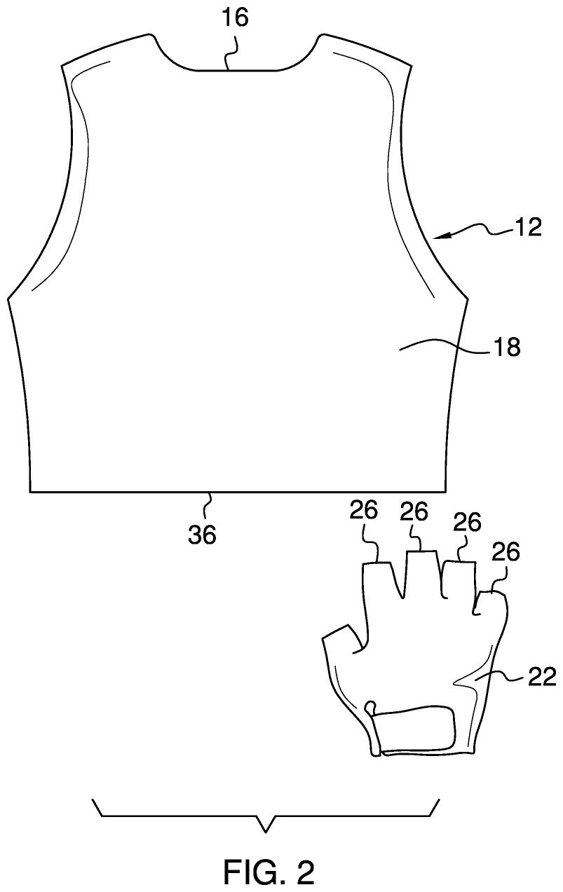

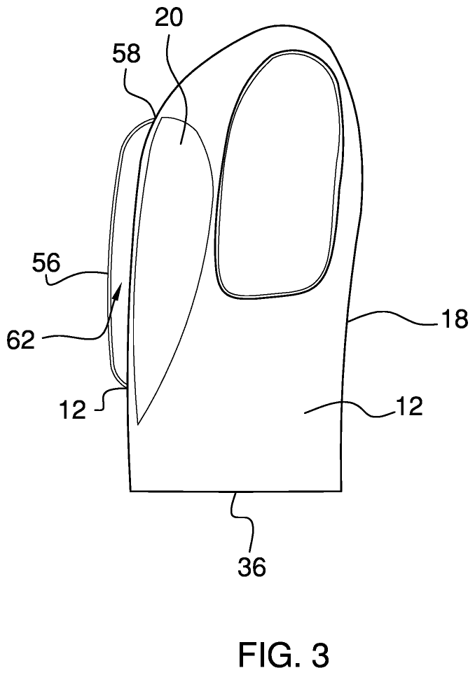

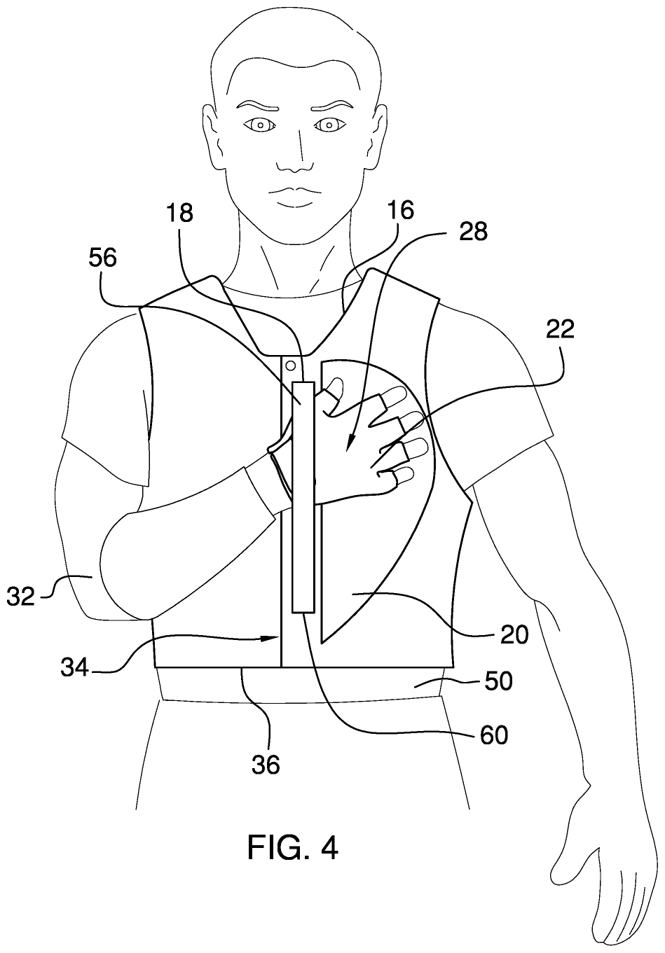

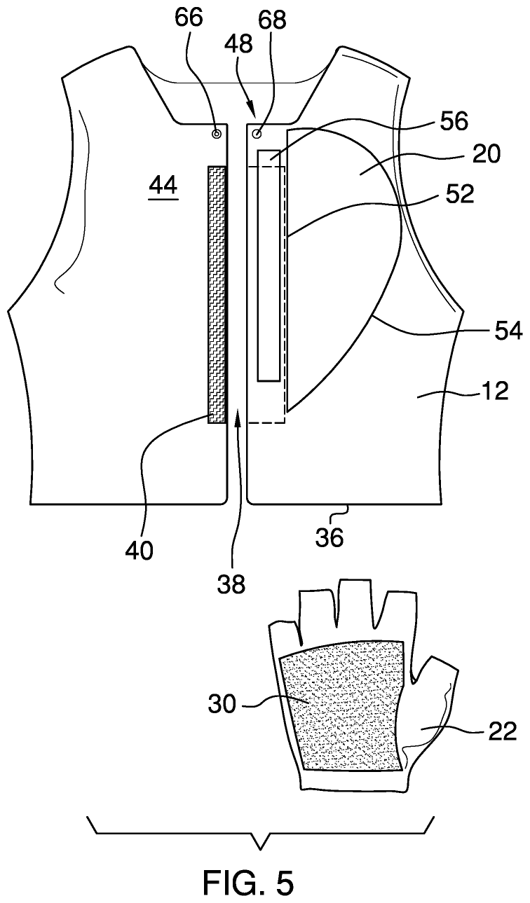

With reference now to the drawings, and in particular to FIGS. 1 through 5 thereof, a new sleep garment device embodying the principles and concepts of an embodiment of the disclosure and generally designated by the reference numeral 10 will be described.

As best illustrated in FIGS. 1 through 5, the arm immobilizing sleep garment device 10 generally comprises a vest 12 having a chest 14, a neck 16, and a back 18. A mating panel 20 is coupled to the chest 14 of the vest 12. A glove 22 has a palm 24 and a plurality of finger sleeves 26 wherein the glove 22 is configured for being worn on a hand 28. A mating piece 30 is coupled to the glove 22. The mating piece 30 is complementary to the mating panel 20 wherein abutting the mating piece 30 to the mating panel 20 engages the glove 22 to the vest 12 such that the glove 22 and vest 12 are configured for immobilizing an arm 32 extending from the hand 28 in the glove 22. The mating panel 20 may have a straight edge 52 and an arcuate edge 54. Opposite ends of the arcuate edge 54 meet the straight edge 52. The mating panel 20 has a maximum width nearer a top of the mating panel 20 than a bottom of the mating panel 20.

A break 34 extends through the vest 12 from the neck 16 to a bottom edge 36. The break 34 is positioned on and divides the chest 14. The mating panel 20 is positioned on one side of the break 34. A vest closure 38 is coupled to the chest 14 of the vest 12. The vest closure 38 includes a first portion 40 of hook and loop fastener 42 coupled to an exterior surface 44 of the chest 14 adjacent to the break 34. A second portion 46 of hook and loop fastener 42 is coupled to an interior surface 48 of the chest 14 adjacent to the break 34 on an opposite side of the break 34 from the first portion 40 of hook and loop fastener 42 wherein the second portion 46 of hook and loop fastener 42 is positionable to overlap the first portion 40 of hook and loop fastener 42. The second portion 46 of hook and loop fastener 42 is complementary to the first portion 40 of hook and loop fastener 42. Thus, the first 40 and second 46 portions of hook and loop fastener 42 are engageable to each other to close the break 34. The first portion 40 and second portion 46 of hook and loop fastener 42 may be sized and positioned to allow for engagement when the vest 12 is snugly wrapped around a torso 50. The mating panel 20 is positioned on the chest 14 on a same side of the break 34 as the second portion 46 of hook and loop fastener 42. Thus, the break 34 can be closed without any portion of the vest 12 overlapping and obscuring access to the mating panel 20.

An arm band 56 has a top end 58 and a bottom end 60. The top end 58 is coupled to the chest 14 of the vest 12 adjacent to the break 34. The bottom end 60 is coupled to the chest 14 of the vest 12 adjacent to the break 34 and spaced below the top end 58 wherein the arm band 46 forms a loop 62 positioned on the chest 14 extending parallel to the break 34. The arm band 56 is configured for receiving the arm 32 through the loop 62. Thus, the loop 62 is configured to facilitate holding the arm 32 proximate to the vest 12 while the glove 22 is engaged to the mating panel 20. The arm band 56 is positioned between the mating panel 20 and the break 34.

A neck closure 64 coupled to the vest 12. The neck closure 64 includes a first snap fastener 66 coupled to the exterior surface 44 of the chest 14 adjacent to the break 34 and the neck 16 of the vest 12. A second snap fastener 68 is coupled to the chest 14 adjacent to the break 34 and the neck 16 on an opposite side of the break 34 from the first snap fastener 66 wherein the second snap fastener 68 is positionable to overlap the first snap fastener 66. The second snap fastener 68 is complementary to the first snap fastener 66 such that the first 66 and second 68 snap fasteners are engageable to each other to close the break 34 adjacent to the neck 16 of the vest 12. It is specifically contemplated that equivalent fasteners to snap fasteners may be used such as hook and loop fastener, hook and eye, or the like.

In use, the vest 12 is positioned on the torso. The neck closure 64 and vest closure 38 are used to secure the vest 12 on the torso. The glove 22 is positioned on the hand 28. The arm 32 is inserted through the loop 62 and the mating piece 30 is engaged to the mating panel 20 immobilizing the arm 32.

The arm immobilizing sleep garment device 10 is shown for immobilizing a right arm in the drawing figures. However, the arm immobilizing sleep garment device 10 may be produced for immobilizing either a right or left arm by reversing the positions of the vest closure 38, mating panel 20, loop 62 and neck closure 64 relative to the break 34.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An arm immobilizing sleep garment device comprising:
a vest having a chest, a neck, and a back;
a mating panel coupled to said chest of said vest;
a glove having a palm and a plurality of finger sleeves wherein said glove is configured for being worn on a hand; and
a mating piece coupled to said glove, said mating piece being complementary to said mating panel wherein abutting said mating piece to said mating panel engages said glove to said vest such that said glove and vest are configured for immobilizing an arm extending from the hand in the glove.

2. The arm immobilizing garment device of claim 1, further comprising a break extending through said vest, said break being positioned on and dividing said chest.

3. The arm immobilizing garment device of claim 2, further comprising said mating panel being positioned on one side of said break.

4. The arm immobilizing garment device of claim 2, further comprising:
a vest closure coupled to said chest of said vest, said closure comprising
a first portion of hook and loop fastener coupled to an exterior surface of said chest adjacent to said break, and
a second portion of hook and loop fastener coupled to an interior surface of said chest adjacent to said break on an opposite side of said break from said first portion of hook and loop fastener wherein said second portion of hook and loop fastener is positionable to overlap said first portion of hook and loop fastener, said second portion of hook and loop fastener being complementary to said first portion of hook and loop fastener whereby said first and second portions of hook and loop fastener are engageable to each other to close said break.

5. The arm immobilizing garment device of claim 4, further comprising said mating panel being positioned on said chest on a same side of said break as said second portion of hook and loop fastener.

6. The arm immobilizing garment device of claim 1, further comprising an arm band having a top end and a bottom end, said top end coupled to said chest of said vest, said bottom end being coupled to said chest of said vest spaced below said top end wherein said arm band forms a loop positioned on said chest configured for receiving the arm through the loop whereby the loop is configured to facilitate holding the arm proximate to the vest while the glove is engaged to the mating panel.

7. The arm immobilizing garment device of claim 6, further comprising said arm band being positioned between said mating panel and a center of said chest of said vest.

8. The arm immobilizing garment device of claim 2, further comprising an arm band having a top end and a bottom end, said top end coupled to said chest of said vest adjacent to said break, said bottom end being coupled to said chest of said vest adjacent to said break and spaced below said top end wherein said arm band forms a loop positioned on said chest extending parallel to said break, said arm band being configured for receiving the arm through the loop whereby the loop is configured to facilitate holding the arm proximate to the vest while the glove is engaged to the mating panel.

9. The arm immobilizing garment device of claim 8, further comprising said arm band being positioned between said mating panel and said break.

10. The arm immobilizing garment device of claim 4, further comprising an arm band having a top end and a bottom end, said top end coupled to said exterior surface of said chest of said vest adjacent to said break on a same side of said break as said second portion of hook and loop fastener, said bottom end being coupled to said exterior surface of said chest of said vest adjacent to said break and spaced below said top end wherein said arm band forms a loop positioned on said exterior surface of said chest extending parallel to said break, said arm band being configured for receiving the arm through the loop whereby the loop is configured to facilitate holding the arm proximate to the vest while the glove is engaged to the mating panel.

11. The arm immobilizing garment device of claim 10, further comprising said arm band being positioned between said mating panel and said break.

12. The arm immobilizing garment device of claim 2, further comprising a neck closure coupled to said vest, said neck closure comprising
a first snap fastener coupled to an exterior surface of said chest adjacent to said break and said neck of said vest, and
a second snap fastener coupled to said chest adjacent to said break and said neck on an opposite side of said break from said first snap fastener wherein said second snap fastener is positionable to overlap said first snap fastener, said second snap fastener being complementary to said first snap fastener whereby said first and second snap fasteners are engageable to each other to close said break adjacent to said neck of said vest.

13. An arm immobilizing sleep garment device comprising:
a vest having a chest, a neck, and a back;
a mating panel coupled to said chest of said vest;
a glove having a palm and a plurality of finger sleeves wherein said glove is configured for being worn on a hand;
a mating piece coupled to said glove, said mating piece being complementary to said mating panel wherein abutting said mating piece to said mating panel engages said glove to said vest such that said glove and vest are configured for immobilizing an arm extending from the hand in the glove;
a break extending through said vest, said break being positioned on and dividing said chest, said mating panel being positioned on one side of said break.
a vest closure coupled to said chest of said vest, said closure comprising
a first portion of hook and loop fastener coupled to an exterior surface of said chest adjacent to said break, and
a second portion of hook and loop fastener coupled to an interior surface of said chest adjacent to said break on an opposite side of said break from said first portion of hook and loop fastener wherein said second portion of hook and loop fastener is positionable to overlap said first portion of hook and loop fastener, said second portion of hook and loop fastener being complementary to said first portion of hook and loop fastener whereby said first and second portions of hook and loop fastener are engageable to each other to close said break, said mating panel being positioned on said chest on a same side of said break as said second portion of hook and loop fastener;
an arm band having a top end and a bottom end, said top end coupled to said exterior surface of said chest of said vest adjacent to said break on a same side of said break as said second portion of hook and loop fastener, said bottom end being coupled to said exterior surface of said chest of said vest adjacent to said break and spaced below said top end wherein said arm band forms a loop positioned on said exterior surface of said chest extending parallel to said break, said arm band being configured for receiving the arm through the loop whereby the loop is configured to facilitate holding the arm proximate to the vest while the glove is engaged to the mating panel; and
a neck closure coupled to said vest, said neck closure comprising
a first snap fastener coupled to an exterior surface of said chest adjacent to said break and said neck of said vest, and
a second snap fastener coupled to said chest adjacent to said break and said neck on an opposite side of said break from said first snap fastener wherein said second snap fastener is positionable to overlap said first snap fastener, said second snap fastener being complementary to said first snap fastener whereby said first and second snap fasteners are engageable to each other to close said break adjacent to said neck of said vest.
